(19) United States
(12) Patent Application Publication
(10) Pub. No.: US 2017/0231738 A1
(43) Pub. Date: Aug. 17, 2017

(54) IMPLANTABLE ELECTRONIC DEVICE

(71) Applicant: Boston Scientific Scimed, Inc., Maple Grove, MN (US)

(72) Inventor: Gregg Severson, Prior Lake, MN (US)

(21) Appl. No.: 15/429,531

(22) Filed: Feb. 10, 2017

Related U.S. Application Data
(60) Provisional application No. 62/293,916, filed on Feb. 11, 2016.

(51) Int. Cl. A61F 2/00

(52) U.S. Cl. CPC A61F 2/004; A61F 2250/0002; A61F 2210/0014; A61F 2250/0069

(57) ABSTRACT

An implantable medical device includes an electronic control system and an actuator. The actuator changes shape in response to a command or action of the electronic control system to treat an ailment of a person.

### CROSS-REFERENCE TO RELATED APPLICATIONS

This application claims the benefit of priority to U.S. Provisional Application Ser. No. 62/293,916, filed Feb. 11, 2016, the disclosure of which is herein incorporated by reference in its entirety.

### TECHNICAL FIELD

Embodiments of the present invention relate generally to medical devices and, more particularly, to systems and methods for implantable medical devices.

### BACKGROUND

Fecal and urinary incontinence may be caused by weakened sphincter muscles that are no longer capable of holding urine in the bladder or fecal bowel matter in the intestines without leakage or accidental discharge. Existing devices that treat these ailments require use of a manually operated pump to pump fluid from a reservoir into a cuff configured to be disposed around the urethra or rectum. A similar device may be used to treat erectile dysfunction via use of bladder tube actuator(s) implanted inside the penis. These implanted, manually operated pumps may be difficult to locate, orientate, and operate and may be irritating, uncomfortable, or embarrassing, and require additional implantable parts, tubing, etc., that may make implantation and use more difficult or risky and offer additional failure modes of malfunction during use. A need therefore exists for a more reliable, convenient, and discreet device that is user friendly, comfortable, and eliminates the disadvantages of manual control.

### SUMMARY

Various embodiments of the present invention include an implantable electronic control system that controls an actuator. The actuator may be a cuff or clamshell configured to be disposed around the urethra or rectum, a valve inside the urethra or rectum, or one or more bladder tube actuators implanted inside the penis. The actuator changes shape in response to signals from the electronic control system. The signal may be fluid pumped to or from a reservoir into or out of the actuator; in other embodiments, the signal is a current, voltage, or field that changes the shape of the actuator.

In one aspect, an implantable medical device includes an electronic control system comprising a processor for executing computer instructions and an actuator configured for changing shape in response to a command or action of the electronic control system to treat an ailment of a person. One more of the following features may be included with or incorporated into the medical device. A wireless charging pad may be configured to be disposed beneath a surface of the person's skin. The actuator may include a clamshell or cuff configured to be disposed around a urethra or rectum, a valve configured to be disposed in a urethra or rectum, or a bladder-tube configured to be disposed in a penis; the ailment may include urinary incontinence, fecal incontinence, or erectile dysfunction. A battery may supply power to the electronic control system. A wired charging port may be configured to be disposed through a surface of the person's skin. A pump may be configured for pumping fluid into and out of the actuator; a fluid reservoir may be configured for providing fluid to be pumped into the actuator and for receiving fluid pumped out of the actuator. An electronic device may be in wireless communication with the electronic control system. The medical device may include a nerve stimulator, and the actuator may include an electroactive polymer (“EAP”).

In another aspect, an implantable medical device includes an electronic control system comprising a processor for executing computer instructions and a wireless charging pad disposed beneath a surface of the person's skin configured for receiving wireless power and for providing power to the electronic control system, or for recharging battery(s) contained within. An actuator may be configured for changing shape in response to a command or action of the electronic control system to treat an ailment of a person; the actuator may include a clamshell or cuff configured to be disposed around a urethra or rectum, a valve configured to be disposed in a urethra or rectum, or a bladder-tube disposed in a penis. A wired charging port may be configured to be disposed through a surface of the person's skin.

In another aspect, an implantable medical device includes an electronic control system comprising a processor for executing computer instructions and an actuator configured for changing shape in response to an electrical signal of the electronic control system to treat an ailment of a person.

The actuator includes an electroactive polymer (“EAP”) and may be a clamshell or cuff configured to be disposed around a urethra or rectum, a valve configured to be disposed in a urethra or rectum, or a bladder-tube configured to be disposed in a penis. A wireless charging pad may be configured to be disposed beneath a surface of the person's skin, and a wired charging port may be configured to be disposed through a surface of the person's skin.

A rechargeable battery may be used to supply power to the electronic control system and/or actuator and a capacitor may be utilized in conjunction with the battery to energize the actuators with a higher voltage for faster response. In some embodiments, a wireless charging pad is configured to be disposed underneath the person's skin and allows more stronger and reliable wireless recharging; in other embodiments, a wired port is installed through the person's skin. The person may communicate with the electronic control system using an electronic device via either a wireless or wired link.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 illustrates an implantable medical device;

FIG. 2A illustrates an implantable medical device having a pump and fluid reservoir;

FIGS. 2B and 2C illustrate design and operation of a pump;

FIG. 3 illustrates an implantable medical device having a wireless charging pad;

FIG. 4 illustrates an implantable medical device having a wired charging port;

FIG. 5 illustrates an implantable medical device and an electronic device;

FIG. 6A illustrates an implantable medical device having a rectal-cuff actuator;

FIG. 6B illustrates an implantable medical device having a urethral-cuff actuator;

FIG. 6C illustrates an implantable medical device having a bladder-tube actuator;

FIGS. 6D-6G illustrate embodiments of a bladder-tube actuator;

FIG. 6H illustrates layers in a bladder-tube actuator;

FIGS. 6I and 6J illustrate cross-sectional views of a bladder-tube actuator;

FIG. 6K illustrates a clamshell actuator;

FIG. 6L illustrates a urethral valve actuator;

FIG. 7 illustrates an implantable medical device having a nerve blocker or nerve stimulator leads; and

FIG. 8 illustrates an electronic control system.

### DETAILED DESCRIPTION

Various embodiments of the present invention include systems and methods for treating medical conditions, such as urinary incontinence, fecal incontinence, erectile dysfunction, or other medical conditions. An implantable electronic control system is powered by an implantable battery and optional capacitor that control an implantable actuator, such as a clamshell or cuff disposed around a person's urethra or rectum; or an internal implanted valve disposed inside a person's urethra or rectum, and/or a bladder-tube actuator disposed in a person's penis. Upon receiving a signal from the person via a button or similar feature disposed on and/or electrically connected to the electronic control system and/or via an electronic device in wired or wireless communication with the electronic control system, the electronic control system causes the actuator to change shape by, for example, contracting, bending, expanding, deflating, etc. The clamshell or cuff actuator may change shape to allow, restrict, prevent or otherwise control flow of urine through a urethra or flow of fecal matter through a rectum; the bladder-tube actuator may change shape to inflate and deflate to cause the patient's penis to become erect or flaccid; and the valve may change shape to allow or permit, restrict, prevent or otherwise control flow of urine through the urethra. The actuator may change shape in response to fluid being pumped into it or out of it with a pump; the fluid may be pumped from the actuator to a reservoir or from the reservoir to the actuator. The fluid may be saline or any other fluid. In other embodiments, the actuator includes a material that changes shape in response to an electric current and/or voltage, electric, magnetic, and/or electromagnetic field, or other such effect or force, such as electroactive polymer (“EAP”), a shape-memory alloy, a piezoelectric material, or any other combination or composite of different materials. The actuator may be in the form of an artificial muscle wire, sheet, laminate material, or any other form. In some embodiments, the actuator is “open” or “on” when an electrical signal is applied to it and “closed” or “off” when the electrical signal is unapplied.

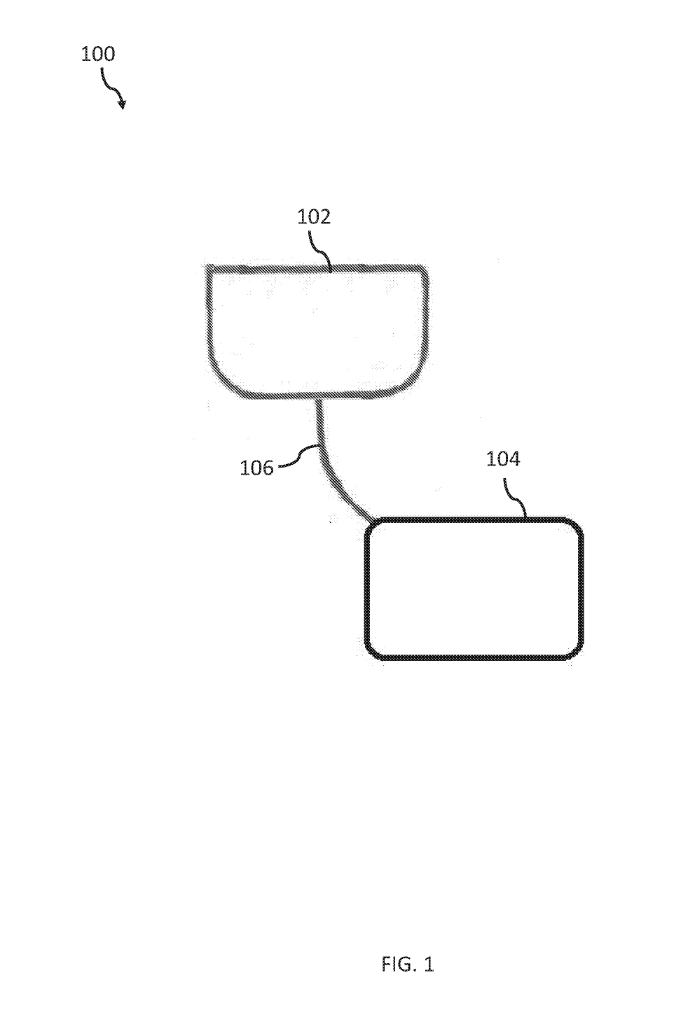

FIG. 1 illustrates a medical device 100 in accordance with various embodiments of the present invention. An electronic control system 102 and an actuator 104 are implanted in a person's body. The electronic control system 102 may include a housing that surrounds and protects one or more electronic components; the actuator 104 may be a clamshell, cuff, bladder tube actuator, valve, or any other similar type of actuator. A connection 106 connects the electronic control system 102 and actuator 104; the connection may include one or more of tubing for carrying a fluid, such as kink-resistant tubing, electrical conductors for carrying analog and/or digital electronic signals, strengthening wires, or any other such material. The electronic control system 102 controls the actuator 104 via the connection 106 by sending and receiving electrical or fluid signals. The actuator 104 may include a sensor, such as a strain/force sensor, that monitors a force applied to the person's body by the actuator and transmits a signal to the electronic control system 102 based thereon. If the sensed force is greater than a threshold force, the electronic control system may adjust the actuator 104 to reduce the applied force.

The housing of the electronic control system 102 may be made using a biocompatible material, such as titanium. The housing may be hermetically sealed; in some embodiments, the housing includes one or more sealed feed-through connectors that provide interconnection with other systems, leads, probes, sensors, etc., as required. The electronic control system 102 may be backfilled with an inert gas (such as argon or nitrogen) to prevent or reduce condensation in its interior. The electronic control system 102 may have a modular design to maximize device packaging density with electronic interconnects to other internal or external system components, i.e., a lithium ion battery, capacitor, and analog, digital, or fiber-optic sensors. The electronic control system 102 may further include any suitable material(s) or coatings, to shield the device from electrical and/or magnetic interference, which may affect wireless communication and signal processing capability.

In various embodiments, the electronic control system 102 includes one or more electronic circuits. These circuits may include a microcontroller, microprocessor, digital signal processor, volatile memory (e.g., random-access memory), non-volatile memory (e.g., read-only memory or flash memory), and/or network transceiver (e.g., a BLUETOOTH or WI-FI transceiver) disposed in or on a circuit board or as a system-on-a-chip or system-in-a-package. The circuits may include digital, analog, and/or mixed-signal circuits.

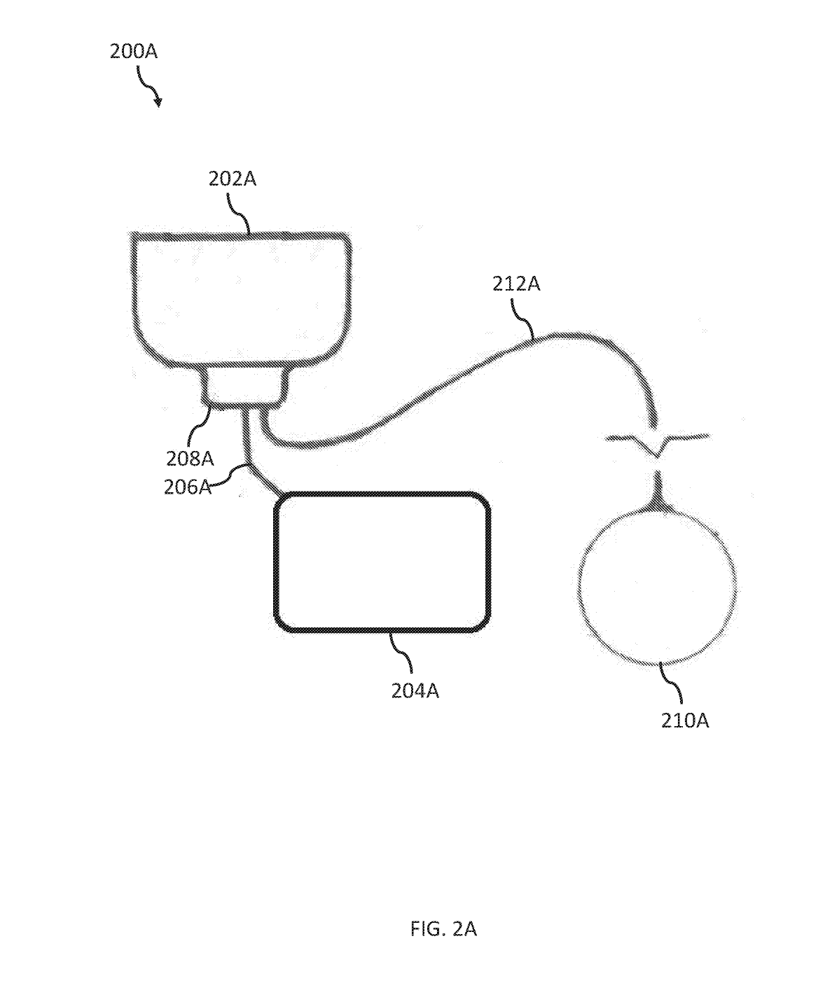

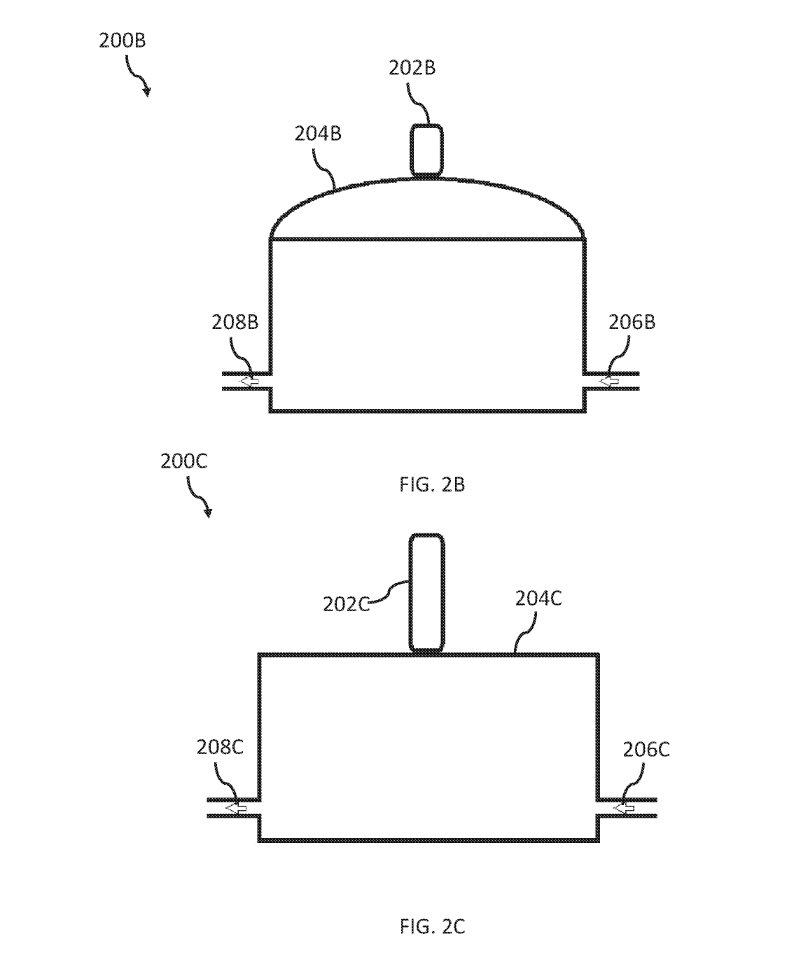

FIG. 2A illustrates a medical device 200A that includes an electronic control system 202A, actuator 204A, and connector 206A. In some embodiments, the actuator 204A changes its shape when a fluid is pumped into it or out of it. The electronic control system 202A may therefore include a pump 208A that pumps fluid into the actuator 204A or out of the actuator 204A; fluid pumped out of the actuator 204A may be stored in a reservoir 210A connected to the pump via tubing 212A. The electronic control system 202A and/or pump 208A may have a pressure relief valve and/or sensor to prevent over-pressuring the system 200A; an overflow valve port may enable excess fluid to flow via tubing to the reservoir 21OA.

The pump 208A may be attached to or otherwise integrated with the electronic control system 202A, and the reservoir may be implanted in a different location in the person's body, as shown in FIG. 2A. The reservoir may be any size, shape, or geometry and be disposed in any location to enable it to be custom fitted to accommodate different physical sizes of people. In other embodiments, the pump 208A is disposed in a location different from that of the electronic control system 202A, and the reservoir is attached to or otherwise integrated with the electronic control system 202A. Any placement or combination of the electronic control system 202A, pump 208A, and reservoir 21OA is, however, within the scope of the present invention.

The pump 208A may be designed to be self-priming using internal fluidic circuitry/logic and may contain check valves, poppet valves, relief valves, and/or pressure/strain gage sensors to monitor and/or control the flow of fluid in the connector 206A. The pump 208A may be a single-stage or two-stage fluid hydraulic pump. Any other type of pump, including a diaphragm, displacement, piston, diastolic, diffusion, centrifugal, spring-loaded solenoid, and/or electro-mechanical pump is within the scope of the present invention. The pump 208A may be operated as a linear pump (e.g., a vertical or horizontal two-way piston pump) or as a rotary pump (e.g., a vane or gear pump). The pump may be constructed using silicone, graphene, or any other material, and may contain rare-earth magnets, piezoelectric elements, MEMS elements, or any other device or material.

In some embodiments, the pump motor is constructed using electroactive polymer (“EAP”) and may exhibit low power consumption, fast response, low noise, low vibration, and/or minimal number of moving parts to thereby maximize device function and reliability. The EAP material may deflect in response to an electric field when, for example, a voltage is applied to the EAP material, causing the actuator to change shape; the EAP material may exhibit a large amount/ratio of deflection (i.e., stroke) with large force, low heat, and/or long life. The types of EAP material may include ionic, dielectric, or ionomeric, and may include any other combination of composite or semiconductor materials.

In some embodiments, the medical device 200A does not include a fluid-based actuator 204A, pump 208A, or reservoir 210A. In these embodiments, the actuator 204A changes shape in response to an applied voltage, current, electric and/or magnetic field, or other force or field. The voltage, current, and/or field may be generated by the electronic control system 202A and transmitted to the actuator 204A via the connection 206A. The actuator 204A may include any substance that changes shape in response to an applied voltage, current, and/or field, such as a piezoelectric element or an EAP element. The EAP element may be constructed from single or multiple layers of EAP material and/or over-molded with other material(s), such as alloys or composites, to provide enhanced properties, texture, and/or biocompatibility.

FIGS. 2B and 2C illustrate pumps 200B, 200C. A pump actuator 202B, 202C is formed of an electrically controllable material, such as EAP, and expands or contracts to move a diaphragm 204A, 204B, which may be made of a flexible material, such as rubber. FIG. 2B illustrates the pump actuator 202B in its contracted state when, e.g., no power is applied to it; FIG. 2C illustrates the pump actuator 202B in its expanded state (e.g., when power is applied to it). The motion of the diaphragm 204B, 204C forces fluid in through a first valve 206B, 206C and out through a second value 208B, 208C. The valves may be check valves or similar valves.

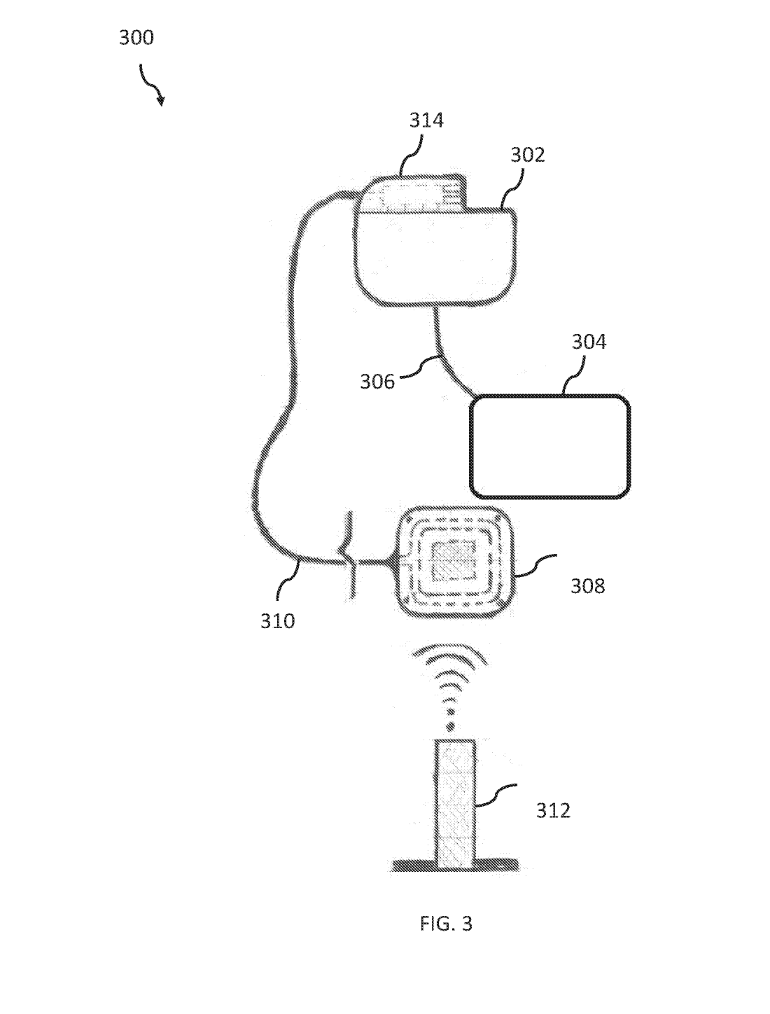

FIG. 3 illustrates a medical device 300 that includes an electronic control system 302, an actuator 304, and a connector 306 therebetween. In some embodiments, the electronic control system 302 includes a rechargeable battery, such as a lithium-ion battery, and capacitor that may be recharged wirelessly. In some embodiments, however, the electronic control system 302 is disposed in a location in the person's body that is not possible or practical for wireless recharging; too much tissue, cartilage or bone for example, may lie between the electronic control system 302 and a wireless power source, making wireless charging weak (slow), impractical or impossible. In some embodiments, a wireless charging pad 308 is connected to the electronic control system 302 via a conductor 310. The wireless charging pad 308 may include conductors arranged to efficiently receive wireless power, such as coils or antennas; the conductors may include copper, silver, gold, or any other conductor or super conductor material(s). The wireless charging pad 308 may further include an internal antenna, RFID circuitry, NFC circuitry, and/or other circuitry for wireless data transmission and communications; this circuitry may permit the medical device 300 to transmit and receive data with another device, such as a mobile device or server. The data may include diagnostic information, use history information, commands, and/or software updates. The wireless charging pad 308 may be any shape, size, and thickness. For example, the wireless charging pad may be flat and flexible, contoured, round and flexible or rigid. The wireless charging pad 308 may be disposed in a location in the person's body such that only a minimal amount of tissue lies between it and a wireless power source 312, such as just under the skin of the person's thigh, stomach, lower back, side, or any other body part. The wireless charging pad 308 could be disposed on the exterior of the body, such as a pad adhered to an exterior portion of skin at the locations mentioned above. The wireless charging pad 308 may be over molded with a biocompatible material, such as silicone, and may include molded holes for the surgeon to secure using sutures to prevent migration.

The conductor 310 may be received directly by the electronic control system 302; in some embodiments, the electronic control system 302 includes an attached connector body 314 that receives the conductor 310. The connector body 314 may be an over-molded connector body that is integrated onto the housing of the electronic control system 302 and sealed off using, for example, over-molding, bonding, interference fit, oil ring seal(s), etc., to prevent moisture intrusion into the electronic control system 302. The connector body 314 may contain integral electrical lead interconnects with redundant molded-in 0-ring seal(s) to prevent moisture intrusion when electrical leads or tubing are engaged into its male and/or female connector sockets. In some embodiments, the connector body 314 is designed such that the conductor 310 is not removable by, for example, hardwiring the conductor 310 and then over-molding, potting, and bonding to prevent moisture intrusion into the device and to provide redundant sealing. The conductor 310 may be shielded to prevent, for example, EMI/RFI interference or to allow the device to be certified as MRI safe to prevent possible harm or discomfort to the person during MRI testing. Wireless power may be provided by a wireless charger 312. In various embodiments, the wireless charger is a dish or other antenna and may be embedded inside a special rug/mat on the floor, hung on the wall, contained within a panel, or inside a waist-band, vest, or other special use clothing worn externally by the user to discreetly re-charge batteries with a more powerful induction current. A portable wireless charger may be, for example, plugged into a cigarette plug power adapter inside an automobile or hardwired into the automobile power system, or embedded within a mat/seat cushion. In further embodiments, wireless charging can be performed using radio frequency (RF) or infrared (IR) lasers to charge the batteries.

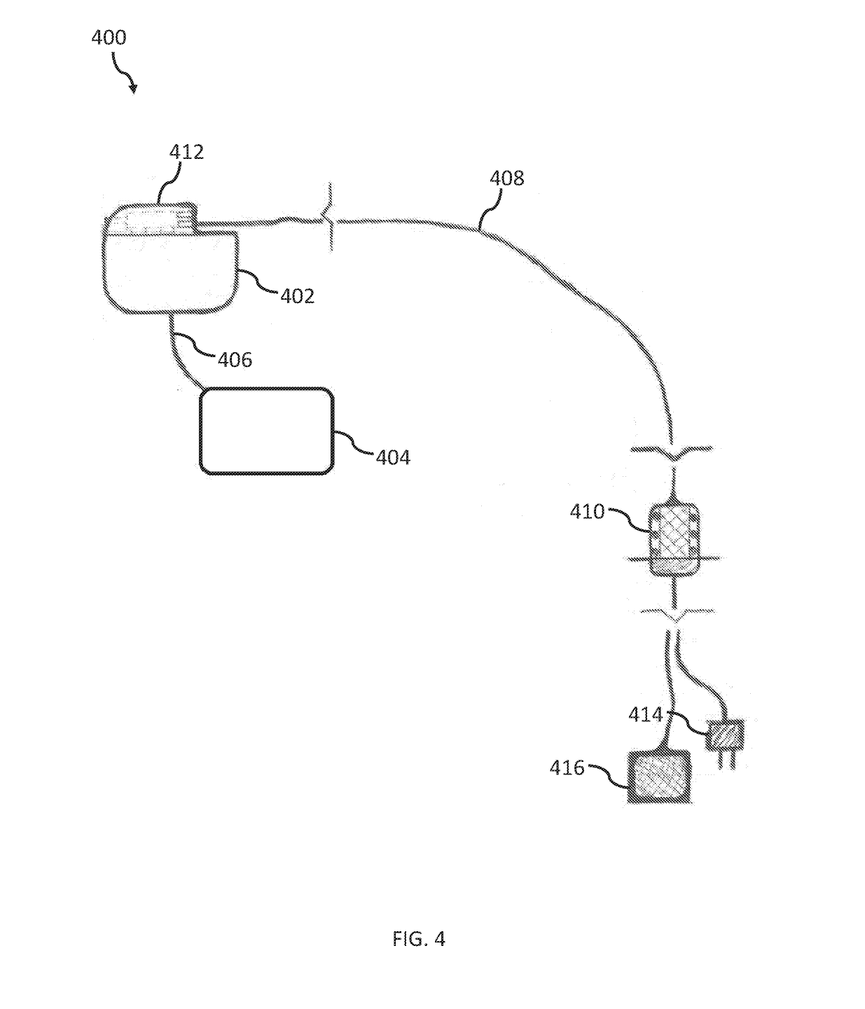

FIG. 4 illustrates a system 400 that includes wired charging of a battery. An electronic control system 402 and an actuator 404 communicate via a connector 406 disposed therebetween. A conductor 408 connects a wired charging port 410 to the electronic control system 402 (via, in some embodiments, a connector body 412). One end of the wired charging port 410 may be disposed outside the skin of the person and may be protected by a sealed connector cap; the other end of the wired charging port 410 may be disposed beneath the skin of the person. The person may remove the sealed connector cap and manually plug a power/charging cable 414, such as a USB cable, into the wired charging port 410 to thereby recharge the battery in the electronic control system 402 via the conductor 408.

The wired charging port 410 may be biocompatible with the person; in some embodiments, the wired charging port 410 is treated so the distal end (with respect to the implantable device) of the wired charging port 410—i.e., the end located outside the skin of the person—promotes permanent tissue growth in/around the outside diameter of the wired charging port 410, thereby sealing off the wired charging port 410 after tissue healing to thereby prevent or reduce bodily fluid leakage and/or risk of infection. The wired charging port 410 may be similar to bulkheads that provide a hermetic feed-through port for connectors and may be also designed for multiple miniaturized connectors, data ports, hoses, or other functions to permit direct user access for monitoring, software updates, diagnostics, metrics, etc. as an alternate to wireless access. The coating material and/or sub-surface material may be modified to optimize the ability to be coated/impregnated with other materials or drugs, i.e., they may be drug-eluting, bio-absorbable, promote tissue growth, and/or reduce risk of infection. In some embodiments, the wired charging port 410 includes hole(s) and/or undercut(s) in its exterior to facilitate implantation and/or recovery, such as holes for sutures.

The wired charging port 410 may be molded or machined from biocompatible polymers, metals, alloys, or composites. In some embodiments, the wired charging port 410 is vacuum potted to seal off any leads and thereby prevent moisture intrusion, which may lead to intermittent device operation or failure. The portion of the wired charging port 410 located above the skin may have a seal cap with redundant molded seals and/or o-rings that may be replaceable after certain amount of time. The portion of the wired charging port 410 disposed below the skin, including conductor leads and/or wires, may be configured to have adequate strain relief before potting, so that any thermal expansion or bend stress does not cause wire breakage or fracture. Mating connector contacts are typically male/female plugs that are designed to reliably mate with constant contact spring force to maintain connectivity over design life. In some embodiments, the female contact plug is exposed once the connector cap is unscrewed or opened, which allows for a low profile to insure it is not damaged.

The battery may be any battery, including non-rechargeable and rechargeable batteries. The battery may be disposed within the electronic control system, within the actuator, or disposed elsewhere in the patient's body. In some embodiments, an external battery back 416 connects to the wired charging port 410 and provides power to the system 400. The external battery back may be used to, for example, allow the person to have a larger power supply for travel if the person does not have access to a power source for charging via the charging cable 414.

FIG. 5 illustrates a system 500 that includes an electronic control system 502 and an actuator 504 that communicate via a connector 506 disposed therebetween. An electronic device 508 communicates wirelessly with the electronic control system 502. The electronic device 508 may be, for example, a smartphone, tablet computer, laptop computer, desktop computer, smart watch or other wearable device, or any other device capable of wireless communication. The wireless link may be BLUETOOTH, WI-FI, NFC, RFID, cellular (e.g., CDMA, GSM, LTE, or similar protocols), or any other protocol. The electronic control system 502 may send and receive wireless signals using an antenna disposed in or on it and/or an antenna disposed elsewhere in the person's body and in electrical communication with the electronic control system 502. In some embodiments, the antenna is included with the wireless charging pad 308 of FIG. 3.

The electronic device 508 may communicate securely with the electronic control system 502 using any of a number of different authentication systems and methods. In some embodiments, the electronic device 508 is connected to the electronic control system 502 via a wired link (such as the cable 408 disclosed above) before it is permitted to communicate wirelessly; the electronic device 508 may exchange security tokens with the electronic control system 502, for example, using the wired link. In other embodiments, the electronic control system 502 is paired with the electronic device 508 before implantation. In some embodiments, the electronic control system 502 includes a unique identifier, such as a string of numbers, letters, or other characters, stored in a read-only memory; this unique identifier may be used to authenticate the electronic device 508 to the electronic control system 502.

Using the electronic device 508, the person may send instructions to the electronic control system 502, receive status information from the electronic control system 502, or perform any other of the functions of the electronic control system 502. For example, the person may command the electronic control system 502 to activate the actuator 504 (i.e., cause the actuator 504 to change shape to constrict the urethra or rectum, close a valve in the urethra or rectum, or cause the penis to become erect) or to de-activate the actuator 504 (i.e., cause the actuator 504 to change shape to relax and allow passage of material through the urethra or rectum or cause the penis to become flaccid). The person may further command the electronic control system 502 to adjust configuration options related to the actuator 504 and/or other parts of the system 500, such as modifying the amount of force applied by the actuator. The status information received from the electronic control system 502 may include remaining battery life, built-in self-test/diagnostic information, usage history (i.e., number and time of activations/de-activations of the actuator 504), authentication status and/or history, firmware/software version/status information, or any other such information. In addition, the electronic device 508 may transmit software/firmware updates to the electronic control system 502 via the wireless and/or wired link.

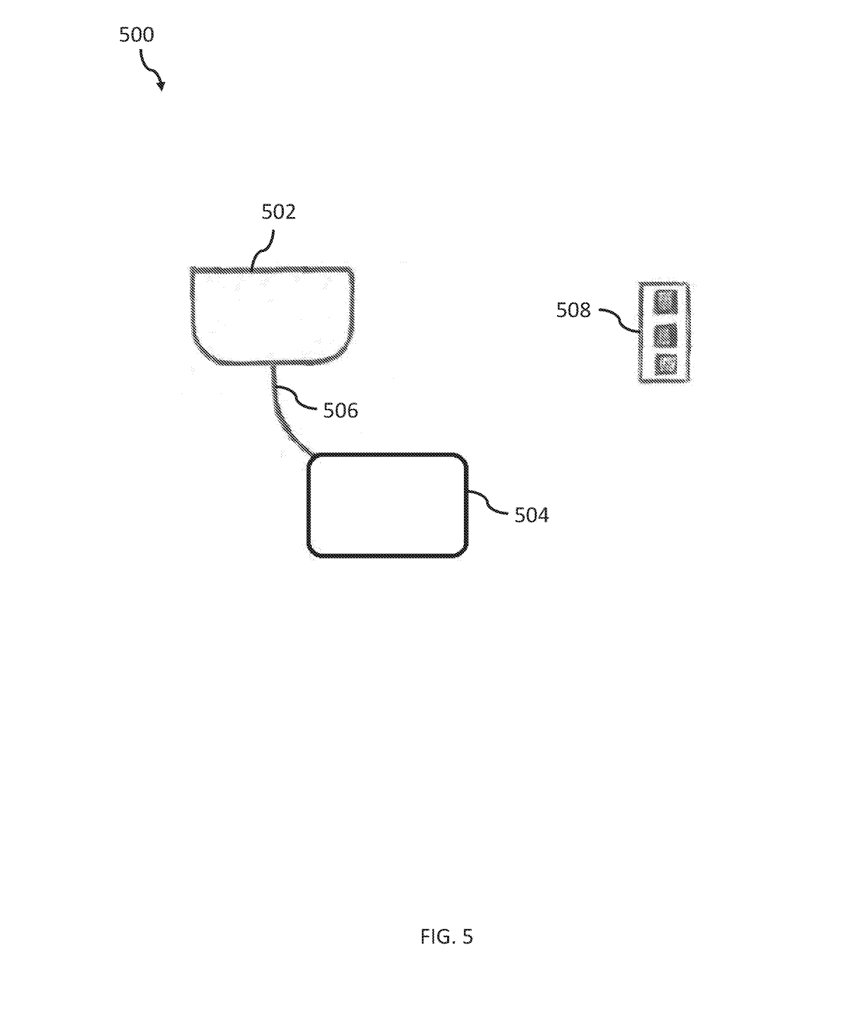

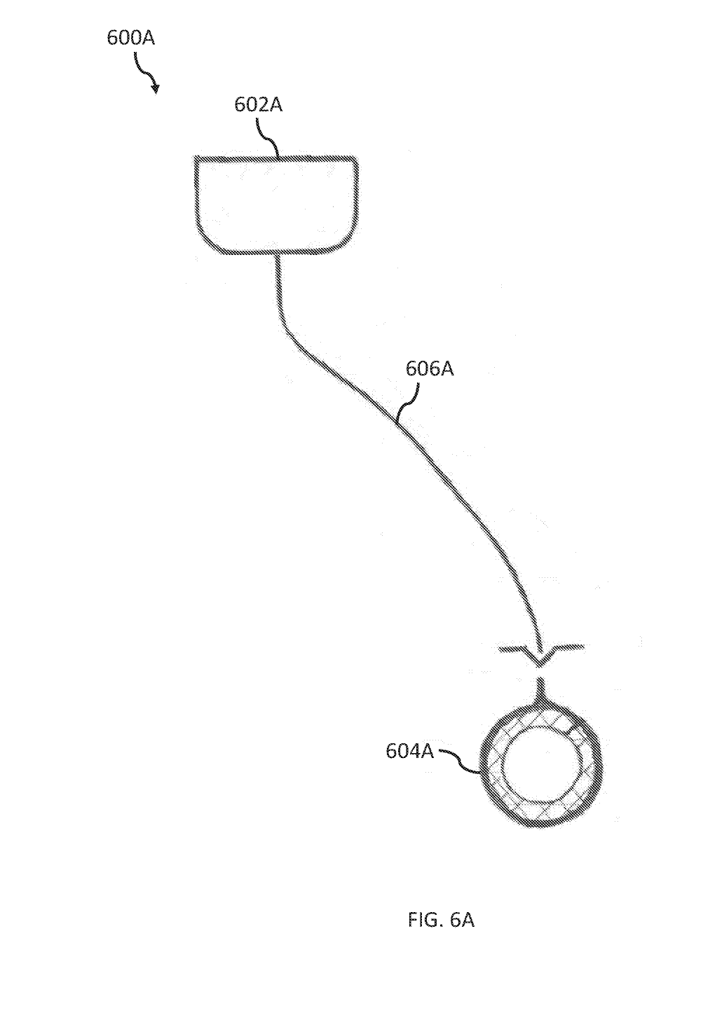

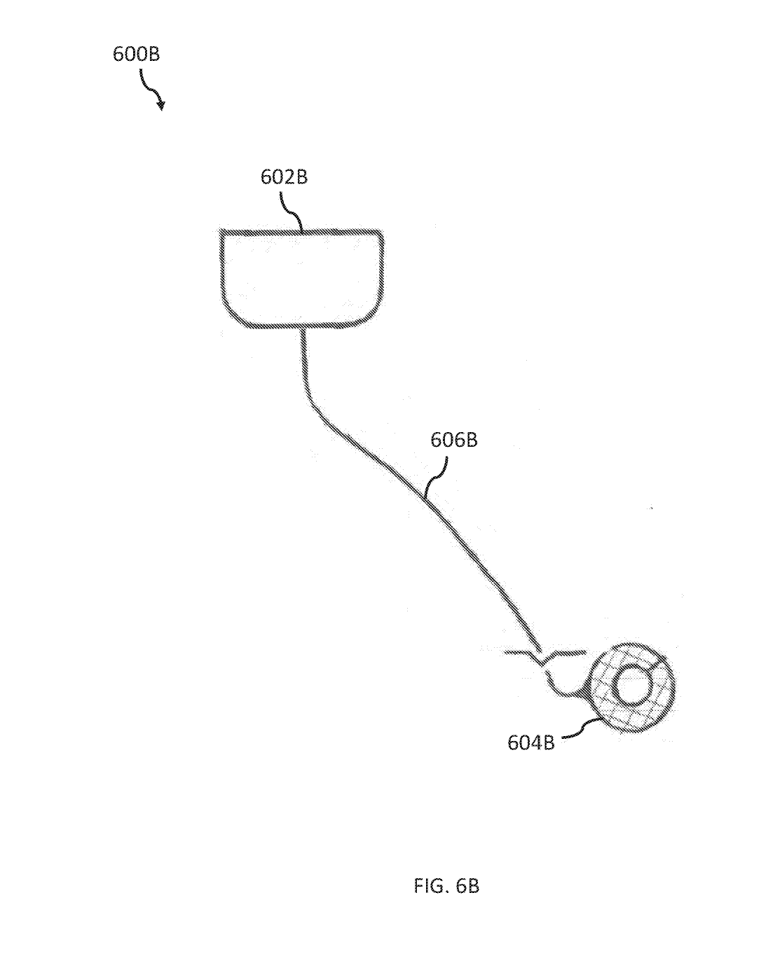

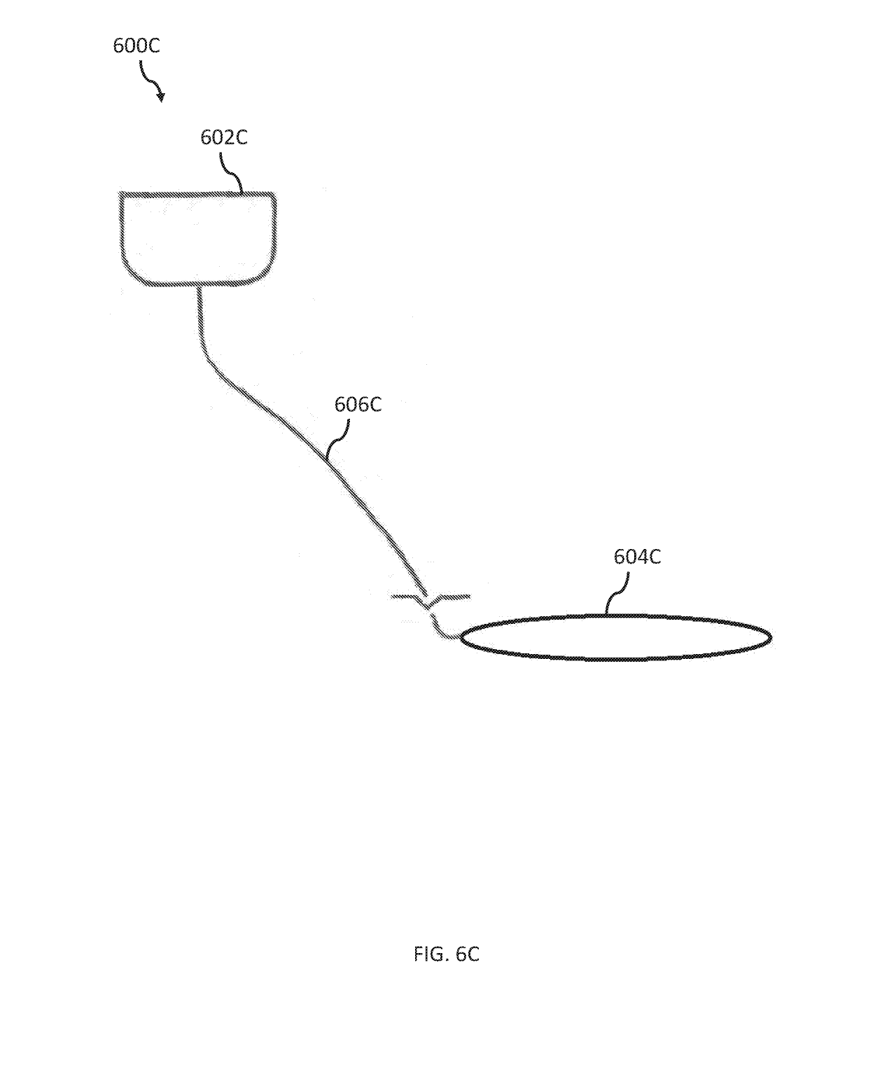

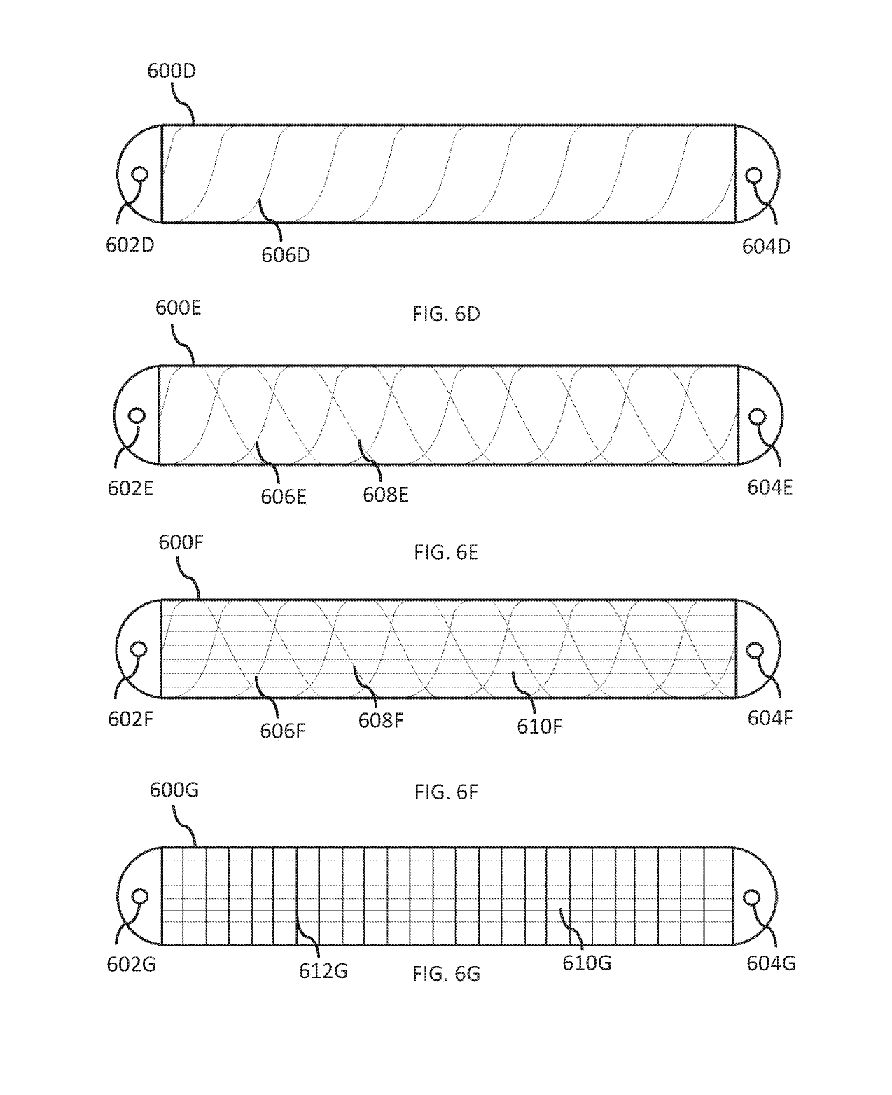

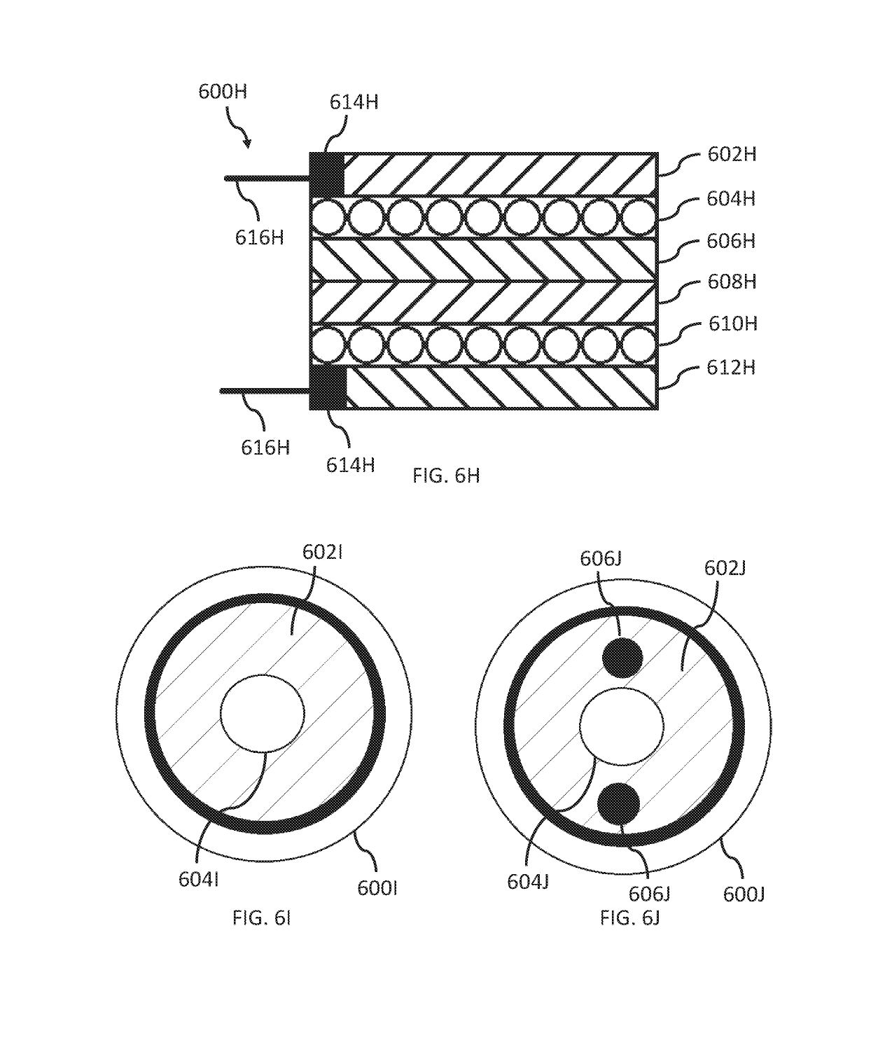

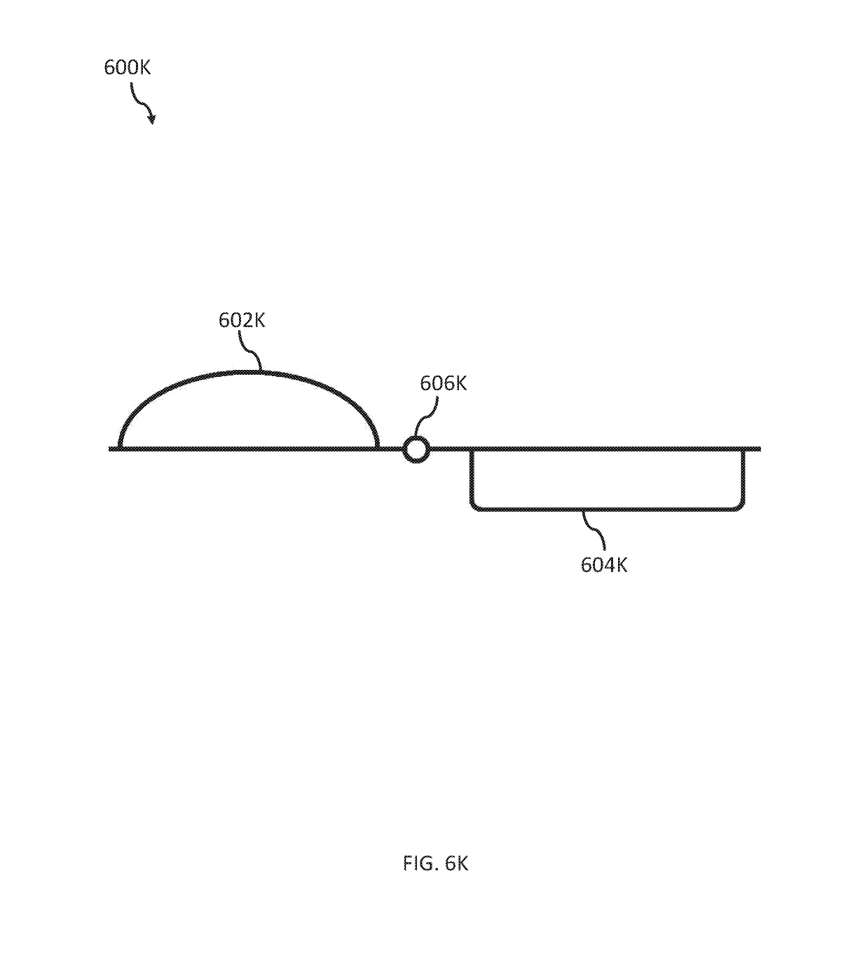

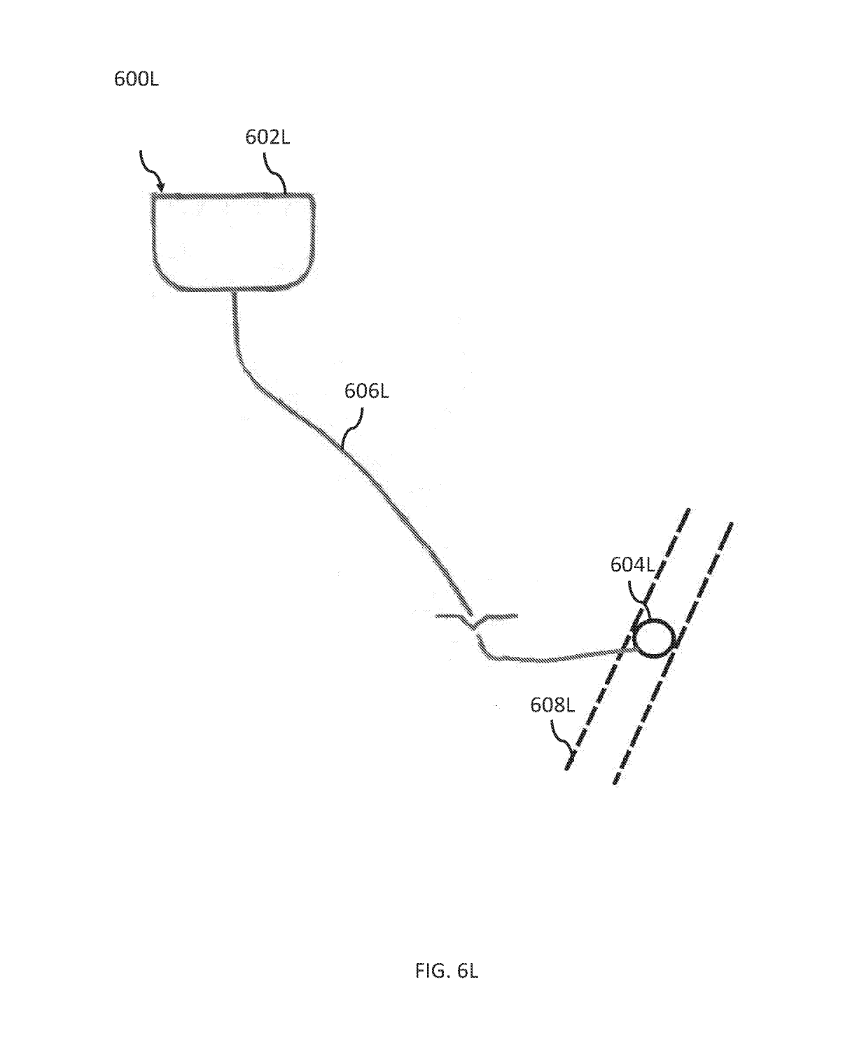

FIGS. 6A, 6B, 6C, 6D, 6E, 6F, 6G, 6H, 6I, 6J, and 6K illustrate exemplary embodiments 600A, 600B, 600C, 600D, 600E, 600F, 600G, 600H, 600I, 600J, 600K, 600L of different types of actuators. FIGS. 6A, 6B, 6C, and 6L include an electronic control unit 602A, 602B, 602C, 602L and a connector 606A, 606B, 606C, 606L. In FIG. 6A, the connector 606A connects a rectal cuff 604A to the electronic control unit 602A. In FIG. 6B, the connector 606B connects a urethral cuff 604B to the electronic control unit 602B. In FIG. 6C, the connector 606C connects a penile bladder-tube 604C to the electronic control unit 602C. In FIG. 6L, the connector 606L connects a urethral valve 604L to the electronic control unit 602L. As mentioned above, the actuators 604A, 604B, 604C, 604L may be controlled via fluid transfer, and the embodiments 600A, 600B, 600C, 600L may include a pump and/or reservoir as described above. Alternatively, or in addition, the actuators 604A, 604B, 604C, 604L may be made of a material that changes shape in response to an electrical current, voltage, or field, such as EAP.

FIGS. 6D, 6E, 6F, and 6G illustrate various embodiments 600D, 600E, 600F, 600G of a bladder-tube actuator. Each embodiment 600D, 600E, 600F, 600G may include holes or orifices 602D, 602E, 602F, 602G at a first end and/or holes or orifices 604D, 604E, 604F, 604G at a second end to aid in attachment to a person's body, such as by using sutures. In FIG. 6D, EAP fibers 606D may be arranged in a helical orientation to increase torsional rigidity. In FIG. 6E, a first set of helical EAP fibers 606E and a second set of helical EAP fibers 608E are included. FIG. 6F adds a set of straight EAP fibers 610F to sets of helical EAP fibers 606D, 606E. In FIG. 6G, the bladder-tube actuator 600G includes straight EAP fibers 610G and circular rings 612G. The EAP fibers may be molded within silicone for biocompatibility. When power is not applied to the EAP fibers, the bladder-tube actuators 600D, 600E, 600F, 600G may be soft and flexible; when power is applied to the EAP fibers, the bladder-tube actuators 600D, 600E, 600F, 600G may be stiff and rigid. In some embodiments, the diameter of the bladder-tube actuators 600D, 600E, 600F, 600G increases when power is applied. The bladder-tube actuators 600D, 600E, 600F, 600G may be filled with an EAP fluid or gel to increase the stiffness and/or diameter of the actuators when power is applied. These orientations of EAP fibers and EAP fluid or gel may be useful as well with respect to the urethral and rectal actuators disclosed herein.

FIG. 6H illustrates a view of the layers 600H in a bladder-tube actuator in some embodiments. A first layer 602H includes a silicon rubber coating; a second layer 604H includes round EAP fibers, which may run longitudinally or axially along the length of the actuator; a third layer 606H includes an EAP composite layer. A fourth layer 608H mirrors the third layer 606H, a fifth layer 610H mirrors the second layer 604H, and a sixth layer 612H mirrors the first layer 602H. Conductors 614H supply power to the actuator via wires 616H. A fabric layer under the silicon rubber layers 602H, 612H may be added to give the EAP material strength and/or protect the EAP material from fold wear or buckling.

FIGS. 6I and 6J illustrate cross-sectional views of actuators 600I, 600J. Each actuator 600I, 600J includes EAP fluid or gel 6021, 602J and a hollow tube 6041, 604J for fluid. Actuator 602J further includes one or more EAP conductors 606J.

FIG. 6K illustrates a clamshell actuator 600K that includes an EAP dome 602K, a lower body 604K, and a hinge 606K. The clamshell actuator 600K is shown in an “open” configuration; in various embodiments, the clamshell actuator 600K is closed, using the hinge 606K, around a urethra or rectum to prevent or allow passage of material therein. When power is applied to the dome 602K, the dome 602K changes shape to defect higher or more outward, and when power is not applied to the dome 602K, it changes shape to deflect lower or less outward. In various embodiments, when power is applied, the dome 602K constricts the urethra or rectum against the lower body 604K to restrict, prevent, or otherwise control the passage of material therethrough.

Referring to FIG. 6L, the urethral valve 604L is disposed in a urethra 608L. The urethral valve 604L may be delivered to the urethra 608L using, for example, via a balloon-catheter delivery system that expands a stent to secure the urinary valve 604L in place. The urethral valve 604D may be made of any material and have any design, such as a ball valve or globe valve design. In some embodiments, the urethral valve 604L is actuated using a magnetic field generated by a coil placed outside the urethra 608L and axially aligned with the urethral valve 604L. In other embodiments, the urethral valve 604L is operated using a self-contained, miniature (micro-actuator) electro-mechanical actuator, i.e., an EAP, piezoelectric, or solenoid motor. Power may be supplied to the urethral valve 604L via induced current or via a power cable. A similar valve set-up configured for the differing anatomy may be implanted internally within the lumen of the rectum.

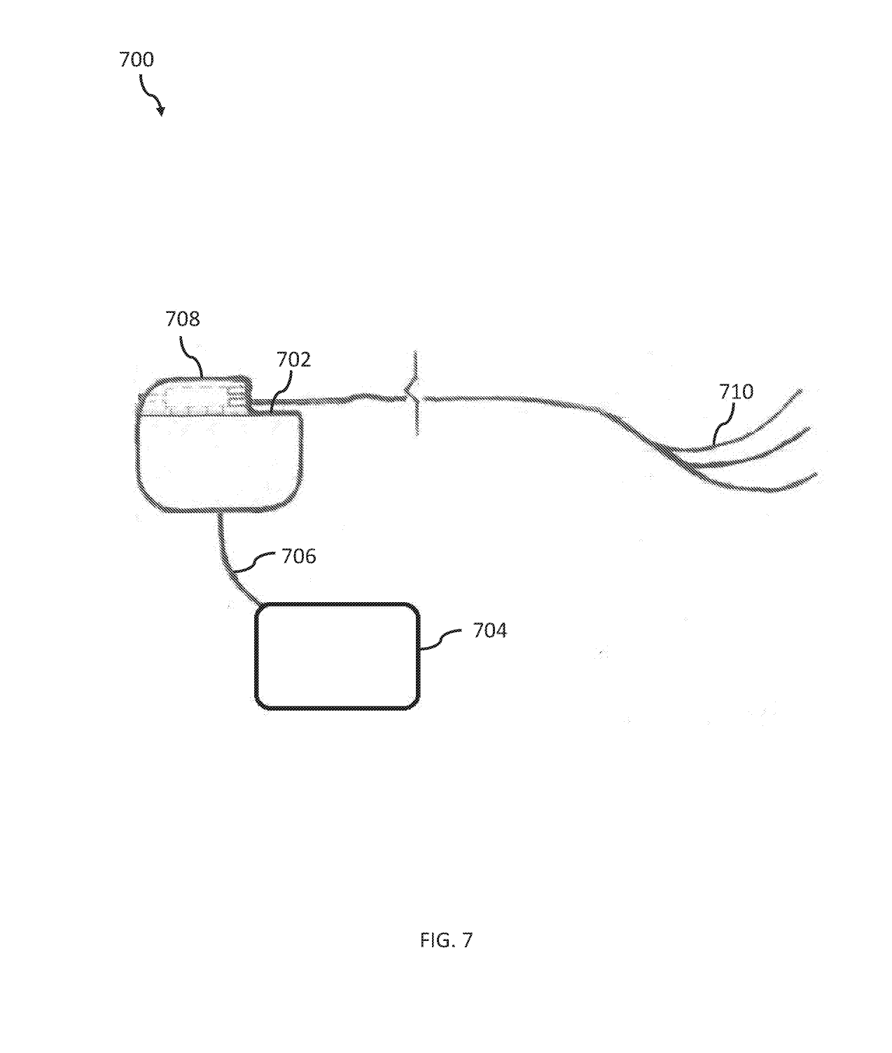

FIG. 7 illustrates a system 700 that includes an electronic control system 702 and an actuator 704 that communicate via a connector 706 disposed therebetween. A connector body 708 is connected to one or more electrical leads 710. An electrical lead 710 may be used to deliver electrical neuro-stimulation or nerve block signals to the person's nerves, such as the sacral nerve. Delivering electrical neuro-stimulation or blocking a nerve to the sacral nerve may serve to reduce the person's urge to urinate or have bowel movements. The electrical lead 710 may be used in conjunction with any of the embodiments described above. The actual application or doctor's prescription for the individual patient may be controlled by software, and any combination of signals may be programmed or sequenced.

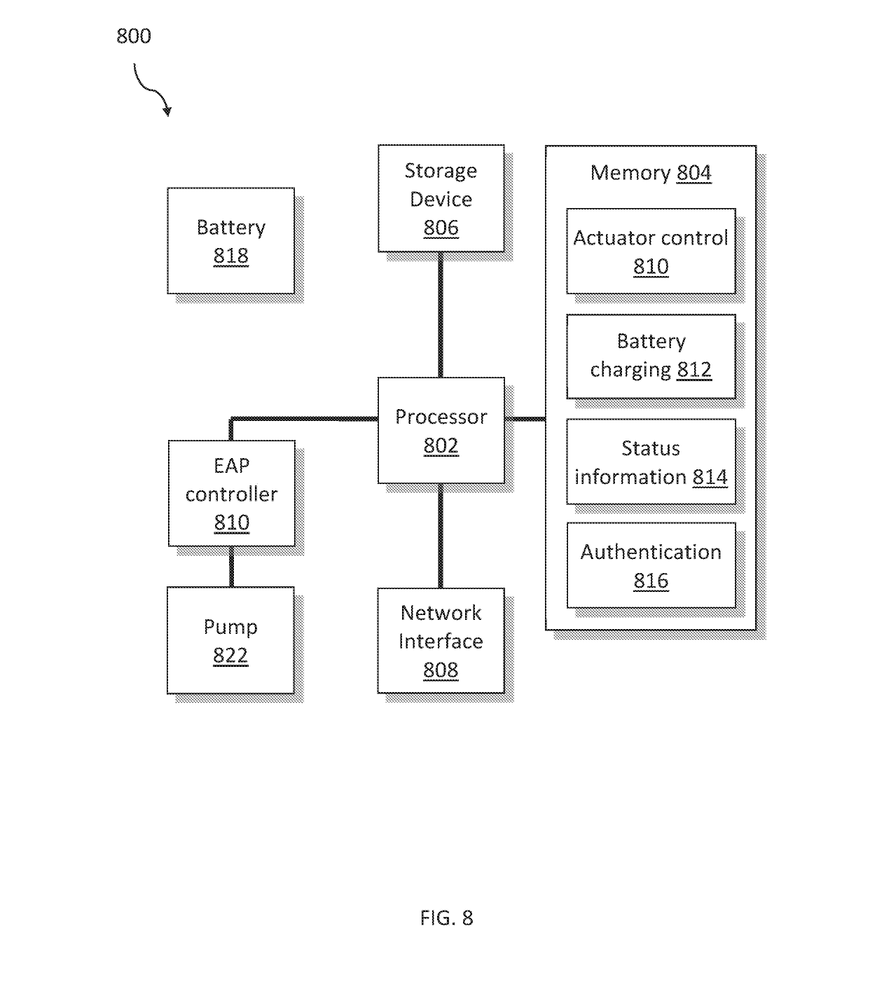

FIG. 8 is a block diagram of an electronic control system 800 implementing embodiments of the present invention. The electronic control system 800 may include a processor 802 having one or more central processing units (CPUs), volatile and/or non-volatile main memory 804 (e.g., RAM, ROM, or flash memory), one or more mass storage devices 806 (e.g., hard disks, or removable media such as CDs, DVDs, USB flash drives, etc. and associated media drivers), a network interface 808 (e.g., a Wi-Fi or ETHERNET port) may be used to connect the electronic control system 500 to another device.

The main memory 804 may be used to store instructions to be executed by the processor 802, conceptually illustrated as a group of modules. These modules may include an actuator controller 810, a battery-charging/capacitor charging controller 812, status information 814, and/or an authentication controller 816. The various modules may be programmed in any suitable programming language, including, without limitation high-level languages such as C, C++, Java, Perl, Python, or Ruby or low-level assembly languages. The memory 804 may further store input and/or output data associated with execution of the instructions as well as additional information.

A non-rechargeable or rechargeable battery 818 may supply power to the components described herein. The system 800 may further include an EAP controller 810 for supplying electrical signals to an EAP-based actuator or a pump 822 for pumping fluid to or from a fluid-based actuator.

The electronic control system 800 is described herein with reference to particular blocks, but this description is not intended to limit the invention to a particular physical arrangement of distinct component parts. The electronic control system 800 is an illustrative example; variations and modifications are possible. A particular implementation may include other functionality not described herein. The computer processor may be a general-purpose microprocessor, but depending on implementation can alternatively be, e.g., a microcontroller, peripheral integrated circuit element, a customer-specific integrated circuit (“CSIC”), an application-specific integrated circuit (“ASIC”), a logic circuit, a digital signal processor (“DSP”), a programmable logic device such as a field-programmable gate array (“FPGA”), a programmable logic device (“PLD”), a programmable logic array (“PLA”), smart chip, or other device or arrangement of devices.

Certain embodiments of the present invention were described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. An implantable medical device comprising:
an electronic control system comprising a processor for executing computer instructions; and
an actuator configured for changing shape in response to a command or action of the electronic control system to treat an ailment of a person.

2. The medical device of claim 1, further comprising a wireless charging pad configured to be disposed beneath a surface of the person's skin.

3. The medical device of claim 1, wherein the actuator comprises a clamshell or cuff configured to be disposed around a urethra or rectum, a valve configured to be disposed in a urethra or rectum, or a bladder tube configured to be disposed in a penis.

4. The medical device of claim 1, wherein the ailment comprises urinary incontinence, fecal incontinence, or erectile dysfunction.

5. The medical device of claim 1, further comprising a battery for supplying power to the electronic control system.

6. The medical device of claim 1, further comprising a wired charging port configured to be disposed through a surface of the person's skin.

7. The medical device of claim 1, further comprising a pump configured for pumping fluid into and out of the actuator.

8. The medical device of claim 7, further comprising a fluid reservoir configured for providing fluid to be pumped into the actuator and for receiving fluid pumped out of the actuator.

9. The medical device of claim 1, further comprising an electronic device in wireless communication with the electronic control system.

10. The medical device of claim 1, further comprising a nerve stimulator.

11. The medical device of claim 1, wherein the actuator comprises an electroactive polymer (“EAP”).

12. An implantable medical device comprising:
an electronic control system comprising a processor for executing computer instructions; and
a wireless charging pad configured to be disposed beneath a surface of the person's skin configured for receiving wireless power and for providing power to the electronic control system.

13. The medical device of claim 12, further comprising an actuator configured for changing shape in response to a command or action of the electronic control system to treat an ailment of a person.

14. The medical device of claim 13, wherein the actuator comprises a clamshell or cuff configured to be disposed around a urethra or rectum, a valve configured to be disposed in a urethra or rectum, or a bladder tube configured to be disposed in a penis.

15. The medical device of claim 12, further comprising a wired charging port configured to be disposed through a surface of the person's skin.

16. An implantable medical device comprising:
an electronic control system comprising a processor for executing computer instructions; and
an actuator configured for changing shape in response to an electrical signal of the electronic control system to treat an ailment of a person.

17. The medical device of claim 16, wherein the actuator comprises an electroactive polymer (“EAP”).

18. The medical device of claim 16, wherein the actuator comprises a clamshell or cuff configured to be disposed around a urethra or rectum, a valve configured to be disposed in a urethra or rectum, or a bladder tube configured to be disposed in a penis.

19. The medical device of claim 16, further comprising a wireless charging pad configured to be disposed beneath a surface of the person's skin.

20. The medical device of claim 16, further comprising a wired charging port configured to be disposed through a surface of the person's skin.
